(19) United States
(12) Patent Application Publication
(10) Pub. No.: US 2025/0032558 A1
(43) Pub. Date: Jan. 30, 2025

(54) ANDROGEN-METABOLIZING LACTIC ACID BACTERIA AND USES THEREOF

(71) Applicant: ACADEMIA SINICA, Taipei (TW)

(72) Inventors: Yin-Ru CHIANG, Taipei (TW); Mei-Jou CHEN, Taipei City (TW); Tsun-Hsien HSIAO, Taipei (TW); Chia-Hung CHOU, Taipei City (TW)

(21) Appl. No.: 18/787,463

(22) Filed: Jul. 29, 2024

Related U.S. Application Data
(60) Provisional application No. 63/516,305, filed on Jul. 28, 2023.

(51) Int. Cl. A61K 35/744; A61K 35/742; A61P 5/26; A61P 17/14

(52) U.S. Cl. CPC A61K 35/744; A61K 35/742; A61P 5/26; A61P 17/14

(57) ABSTRACT

The present disclosure relates to a probiotic composition for use in a method for regulating hyperactivity of a serum androgen level and/or increasing hair re-growth. The present disclosure also relates to a method for treating or preventing a disease, disorder or condition caused by hyperactivity of a serum androgen level.

### PRIORITY INFORMATION

The subject application claims priority to and benefit of U.S. Provisional Patent Application No. 63/516,305, filed Jul. 28, 2023, the content of which is incorporated herein by reference in its entirety.

### SEQUENCE LISTING

The instant application contains a Sequence Listing which is submitted electronically in .xml format and is hereby incorporated by reference in its entirety. The .xml copy created Jul. 26, 2024, is named “G4590-18201US_20240726_SeqListing.xml” and is 13 kilobytes in size.

### FIELD OF THE INVENTION

The present disclosure relates to a field of treatment and/or prevention. Particularly, the present disclosure pertains to new probiotic in reducing androgen and treating and/or preventing hyperandrogenism.

### BACKGROUND OF THE INVENTION

Vertebrates, including humans, can synthesize a variety of sex hormones; however, animals cannot completely degrade these steroids. Sex hormones are recycled between the liver and the gut through enterohepatic circulation; the reabsorption of these steroids occurs mainly in human small intestine as well as rodent caecum. Gut microbes may modify and degrade gut steroids and thus regulate host sex hormone levels and profiles. Abnormally high circulating androgen levels have been considered a causative factor for benign prostatic hypertrophy and prostate cancer in men. Recent animal studies on gut microbiome suggested that gut bacteria are involved in sex steroid metabolism; however, the underlying mechanisms and bacterial taxa remain elusive.

### SUMMARY OF THE INVENTION

The present disclosure is at least based on identification of probiotic and their new applications in reducing circulating androgen levels.

The present disclosure provides a probiotic composition androgen-metabolizing gut bacteria, wherein the androgen-metabolizing gut bacteria comprise comprising Bacillus coagulans and Streptococcus thermophilus, and/or Weissella viridescens and any combination of the above-mentioned bacteria.

In one embodiment, the probiotic composition further comprises Thauera sp.

The embodiments of Bacillus coagulans, Streptococcus thermophilus, Weissella viridescens and Thauera sp. are those described herein.

In one embodiment, the probiotic composition is used as probiotic food or a medicament.

The present disclosure provides a method for treating or preventing a disease, disorder or condition caused by hyperactivity of a serum androgen level and/or increasing hair re-growth in a subject in need thereof, comprising administering androgen-metabolizing gut bacteria to the subject. The present disclosure also provides a use of androgen-metabolizing gut bacteria in the manufacture of a preparation for treating or preventing a disease, disorder or condition caused by hyperactivity of a serum androgen level and/or increasing hair re-growth. Also provided is androgen-metabolizing gut bacteria for use in a method for treating or preventing a disease, disorder or condition caused by hyperactivity of a serum androgen level and/or increasing hair re-growth.

The present disclosure provides a method for regulating a serum androgen level, reducing the circulating dihydrotestosterone level, increasing hair re-growth, and/or treating or preventing hyperandrogenism in a subject, comprising administering androgen-metabolizing gut bacteria to the subject. The present disclosure also provides a use of androgen-metabolizing gut bacteria in the manufacture of a preparation for regulating a serum androgen level, reducing the circulating dihydrotestosterone level, increasing hair re-growth, and/or treating or preventing hyperandrogenism. Also provided is androgen-metabolizing gut bacteria for use in a method for regulating a serum androgen level, reducing the circulating dihydrotestosterone level, increasing hair re-growth, and/or treating or preventing hyperandrogenism.

The present disclosure provides a method for mitigating androgenetic alopecia, benign prostatic hypertrophy and/or prostate cancer in a man subject, comprising administering androgen-metabolizing gut bacteria to the man subject. The present disclosure also provides a use of androgen-metabolizing gut bacteria in the manufacture of a preparation for mitigating androgenetic alopecia, benign prostatic hypertrophy and/or prostate cancer in a man subject. Also provided is androgen-metabolizing gut bacteria for use in a method for mitigating androgenetic alopecia, benign prostatic hypertrophy and/or prostate cancer in a man subject.

The present disclosure provides a method for mitigating polycystic ovary syndrome (PCOS) in a woman subject, comprising administering androgen-metabolizing gut bacteria to the woman subject. The present disclosure also provides a use of androgen-metabolizing gut bacteria in the manufacture of a preparation for mitigating polycystic ovary syndrome in a woman subject. Also provided is androgen-metabolizing gut bacteria for use in a method for mitigating polycystic ovary syndrome in a woman subject.

Examples of the disease, disorder or condition comprise, but are not limited to, androgenetic alopecia, benign prostatic hypertrophy, prostate cancer, polycystic ovary syndrome, acne, hirsutism, hyperandrogenism, male infertility, or seborrhea.

In one embodiment, the androgen-metabolizing gut bacteria can be formulated in a probiotic food or a medicament.

In one embodiment, the androgen-metabolizing gut bacteria is selected from the group consisting of: Bacillus sp., Streptococcus sp. Weissella sp. and any combination thereof.

In one embodiment, the androgen-metabolizing gut bacteria Bacillus sp. is Bacillus coagulans.

In one embodiment, the Streptococcus sp. is Streptococcus thermophilus.

In one embodiment, the Weissella sp. is Weissella viridescens.

In one embodiment, the combination of the bacteria comprises any of the following: Bacillus coagulans and Streptococcus thermophilus; Bacillus coagulans and Weissella viridescens; Streptococcus thermophilus and Weissella viridescens; and Bacillus coagulans, Streptococcus thermophilus and Weissella viridescens.

In one embodiment, the androgen-metabolizing gut bacteria further comprises Thauera sp.

In one embodiment, the androgen-metabolizing gut bacteria is selected from the group consisting of: Bacillus coagulans, Streptococcus thermophilus, Weissella viridescens, Thauera sp. and any combination thereof.

In some embodiments, the combination of the bacteria comprises any of the following: Bacillus coagulans and Thauera sp.; Streptococcus thermophilus and Thauera sp.; Weissella viridescens and Thauera sp.; Bacillus coagulans, Streptococcus thermophiles and Thauera sp.; Bacillus coagulans, Weissella viridescens and Thauera sp.; Streptococcus thermophiles, Weissella viridescens and Thauera sp.; Bacillus coagulans, Streptococcus thermophilus and Weissella viridescens and Thauera sp.

In one embodiment, the Bacillus coagulans disclosed herein is Bacillus coagulans HMB-1 or Bacillus coagulans HMB-2. Bacillus coagulans HMB-1 strain was nationally deposited as BRCAS-C1 under the accession number of NITE ABP-04118 at Patent Microorganisms Depositary, National Institute of Technology and Evaluation (#122, 2-5-8 Kazusakamatari, Kisarazu-shi, Chiba 292-0818, Japan) on May 24, 2024. Bacillus coagulans HMB-2 strain was nationally deposited as BRCAS-C3 under the accession number of NITE ABP-04120 at Patent Microorganisms Depositary, National Institute of Technology and Evaluation on May 24, 2024.

In one embodiment, the Streptococcus thermophilus disclosed herein is Streptococcus thermophilus HMX8-22. This strain was nationally deposited as BRCAS-C2 under the accession number of NITE ABP-04119 at Patent Microorganisms Depositary, National Institute of Technology and Evaluation on May 24, 2024.

In one embodiment, the Weissella viridescens disclosed herein is Weissella viridescens C16-Lac78. This strain was nationally deposited as BRCAS-C4 under the accession number of NITE ABP-04121 at Patent Microorganisms Depositary, National Institute of Technology and Evaluation on May 24, 2024.

In one embodiment, the Thauera sp. disclosed herein is Thauera sp. GDN1.

### BRIEF DESCRIPTION OF DRAWINGS

FIG. 1 shows decrease in androgenic activity through anaerobic DHT metabolism by the lactic acid bacteria (strains BRCAS-C1 to BRCAS-C4). The androgenic activity at Hour 0 was presented as 100%. Symbols represent different bacterial strains and controls: hollow circle, BRCAS-C0 (Thauera sp. GDN1); solid circle, BRCAS-C1 (Bacillus coagulans); solid square, BRCAS-C2 (Streptococcus thermophilus); hollow square, BRCAS-C3 (Bacillus coagulans); hollow triangle, BRCAS-C4 (Weissella viridescens); solid triangle, DHT alone.

FIG. 2 shows administration of the lactic acid bacterium BRCAS-C3 (Bacillus coagulans) may alleviate DHT-induced inhibition of hair re-growth in male mice.

### DETAILED DESCRIPTION OF THE INVENTION

It is to be understood that this invention is not limited to the particular materials and methods described herein. It is also to be understood that the terminology used herein is for the purpose of describing particular embodiments and is not intended to limit the scope of the present invention, which will be limited only by the appended claims.

It must be noted that, as used in this specification and the appended claims, the singular forms “a,” “an,” and “the” include plural referents unless the content clearly dictates otherwise.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the terms “treatment,” “treating,” and the like, cover any treatment of a disease in a mammal, particularly in a human, and include: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “preventing” or “prevention” is recognized in the art, and when used in relation to a condition, it includes administering an agent to reduce the frequency or severity of or to delay the onset of symptoms of a medical condition in a subject, relative to a subject which does not receive the agent.

The term “androgen” is used herein to refer to an agent that has androgenic activity. Androgenic activity may be determined or characterized in any of a variety of ways, including in any of a variety of biological activity assays (e.g., in vitro or in vivo assays, for example utilizing animals and/or animal tissues) in which the agent is observed to have one or more activities similar or comparable to that of a known (i.e., reference) androgen assessed under comparable conditions (whether simultaneously or otherwise). In some embodiments, androgenic activity is or comprises transcriptional regulation (e.g., activation) of an androgen-responsive target gene. In some embodiments, androgenic activity is or comprises binding to an androgen receptor. In some embodiments, androgenic activity is or comprises stimulation of prostate growth in rodents. Exemplary known androgens include, for example, androstanedione, androstenediol, androstenedione, androsterone, dehydroepiandrosterone, dihydrotestosterone (DHT), and testosterone.

The term “hyperandrogenism” as used herein, refers to a condition in which a patient exhibits elevated levels of androgens (e.g., testosterone, androstenedione) in serum.

As used herein, “effective amount” means an amount of an agent to be delivered (e.g., drug, therapeutic agent, diagnostic agent, prophylactic agent, etc.) that is sufficient, when administered to a subject suffering from or susceptible to a disease, disorder, and/or condition, to treat, improve symptoms of, diagnose, prevent, and/or delay the onset of the disease, disorder, and/or condition.

In the present disclosure, “hair loss” refers to a phenomenon of loss of hair from the scalp or thinning of hair, “prevention of hair loss” refers to prevention and inhibition of the hair loss phenomenon, and “promotion of hair growth” refers not only to sprouting of new hair or promotion of the hair sprouting but also to promotion of delay of transition from anagen to catagen in the hair growth cycle, and promotion of healthy growth of existing hair.

As used herein, a “combination” refers to any association between two or among more items. The association can be spatial or refer to the use of the two or more items for a common purpose.

As used herein, a “composition” refers to any mixture of two or more products or bacteria (e.g., agents, modulators, regulators, bacteria etc.). It can be a solution, a suspension, liquid, powder, a paste, aqueous or non-aqueous formulations or any combination thereof. As used herein, the term “pharmaceutical composition” refers to a chemical compound or composition capable of inducing a desired therapeutic effect in a subject. In certain embodiments, a pharmaceutical composition includes an active agent (such as androgen-metabolizing gut bacteria), which is the agent that induces the desired therapeutic effect. In certain embodiments, a pharmaceutical composition includes inactive ingredients such as carriers and excipients.

As used herein, the term “pharmaceutically acceptable formulation” refers to a formulation of androgen-metabolizing gut bacteria that does not significantly abrogate the biological activity, a pharmacological activity and/or other properties of the androgen-metabolizing gut bacteria when the formulated androgen-metabolizing gut bacteria is administered to a subject. In certain embodiments, a pharmaceutically acceptable formulation does not cause significant irritation to a subject.

“Probiotic” means microbial cell preparations or components or metabolites of microbial cells with a beneficial effect on the health or well-being of the host. The definition of probiotic is generally admitted and in line with the WHO definition. The probiotic can comprise a unique strain of micro-organism, a mix of various strains and/or a mix of various bacterial species and genera. In case of mixtures, the singular term “probiotic” can still be used to designate the probiotic mixture or preparation. For the purpose of the present invention, micro-organisms of the genus Lactobacillus are considered as probiotics.

“Prebiotic” generally means a non-digestible food ingredient that beneficially affects the host by selectively stimulating the growth and/or activity of micro-organisms present in the gut of the host, and thus attempts to improve host health.

The present disclosure provides a probiotic composition androgen-metabolizing gut bacteria, wherein the androgen-metabolizing gut bacteria comprise comprising Bacillus coagulans and Streptococcus thermophilus, and/or Weissella viridescens and any combination of the above-mentioned bacteria.

The present disclosure provides a method for treating or preventing a disease, disorder or condition caused by hyperactivity of a serum androgen level and/or increasing hair re-growth in a subject in need thereof, comprising administering androgen-metabolizing gut bacteria to the subject. The present disclosure also provides a use of androgen-metabolizing gut bacteria in the manufacture of a preparation for treating or preventing a disease, disorder or condition caused by hyperactivity of a serum androgen level and/or increasing hair re-growth. Also provided is androgen-metabolizing gut bacteria for use in a method for treating or preventing a disease, disorder or condition caused by hyperactivity of a serum androgen level and/or increasing hair re-growth.

Abnormally high circulating androgen levels, especially serum dihydrotestosterone level, have been considered a causative factor for benign prostatic hypertrophy and prostate cancer in men and polycystic ovary syndrome in women. Host circulating androgen levels may be regulated by androgen-metabolizing gut bacteria, which may then change the host physiology. In one aspect, the present disclosure has identified four lactic acid bacteria, including two strains of Bacillus coagulans, one strain of Streptococcus thermophilus and one strain of Weissella viridescens and microbial physiological investigation indicates their androgen metabolic activity. The present disclosure also confirms that the bacterial strains can increase hair re-growth by reducing the circulating dihydrotestosterone level, mitigate polycystic ovary syndrome in a woman subject and/or mitigate androgenetic alopecia, benign prostatic hypertrophy and/or prostate cancer in a man subject and/or treating or preventing hyperandrogenism.

Examples of the disease, disorder or condition comprise, but are not limited to, androgenetic alopecia, benign prostatic hypertrophy, prostate cancer, polycystic ovary syndrome, acne, hirsutism, hyperandrogenism, male infertility, or seborrhea.

Accordingly, the present disclosure identifies androgen-metabolizing gut bacteria in the following applications: regulating a serum androgen level, reducing the circulating dihydrotestosterone level, increasing hair re-growth, treating or preventing hyperandrogenism, mitigating androgenetic alopecia, benign prostatic hypertrophy and/or prostate cancer in a man and/or mitigating polycystic ovary syndrome in a woman subject.

In one embodiment, the androgen-metabolizing gut bacteria Bacillus sp. is Bacillus coagulans. In one embodiment, the Bacillus coagulans disclosed herein is Bacillus coagulans HMB-1 (BRCAS-C1) or Bacillus coagulans HMB-2 (BRCAS-C3).

In one embodiment, the androgen-metabolizing gut bacteria Streptococcus sp. is Streptococcus thermophilus. In one embodiment, the Streptococcus thermophilus disclosed herein is Streptococcus thermophilus HMX8-22 (BRCAS-C2).

In one embodiment, the androgen-metabolizing gut bacteria Weissella sp. is Weissella viridescens. In one embodiment, the Weissella viridescens disclosed herein is Weissella viridescens C16-Lac78 (BRCAS-C4).

The androgen-metabolizing gut bacteria used in the present disclosure includes, but is not limited to, Bacillus coagulans, Streptococcus thermophilus, Weissella viridescens and any combination thereof.

In one embodiment, the combination of the androgen-metabolizing gut bacteria comprises any of the following: Bacillus coagulans and Streptococcus thermophilus; Bacillus coagulans and Weissella viridescens; Streptococcus thermophilus and Weissella viridescens; and Bacillus coagulans, Streptococcus thermophilus and Weissella viridescens.

In one embodiment, the androgen-metabolizing gut bacteria further comprises Thauera sp. In one embodiment, the Thauera sp. disclosed herein is Thauera sp. GDN1.

Denitrifying betaproteobacteria Thauera spp. are metabolically versatile and often distributed in the animal gut. Thauera sp. strain GDN1 is an unusual betaproteobacterium capable of metabolizing androgen under both aerobic and anaerobic conditions.

In some embodiments, the combination of the androgen-metabolizing gut bacteria comprises any of the following: Bacillus coagulans and Thauera sp.; Streptococcus thermophilus and Thauera sp.; Weissella viridescens and Thauera sp.; Bacillus coagulans, Streptococcus thermophiles and Thauera sp.; Bacillus coagulans, Weissella viridescens and Thauera sp.; Streptococcus thermophiles, Weissella viridescens and Thauera sp.; Bacillus coagulans, Streptococcus thermophilus and Weissella viridescens and Thauera sp.

In some embodiments of the disclosure, the administration of the androgen-metabolizing gut bacteria through oral gavage causes profound effects on the host physiology and gut bacterial community. The results of ELISA assay and metabolite profile analysis indicated an approximately 50% reduction in serum androgen levels in the androgen-metabolizing gut bacteria-administered subject. Moreover, androgenic ring-cleaved metabolites were detected in the fecal extracts of the androgen-metabolizing gut bacteria-administered mice. Furthermore, the RT-qPCR results revealed the expression of the androgen catabolismgenes in the gut of the androgen-metabolizing gut bacteria-administered subject. It is believed, though not intended to be restricted by any theoretical, that the administered androgen-metabolizing gut bacteria regulate serum androgen levels, possibly because it blocked androgen recycling through enterohepatic circulation. Sex steroids serve as a carbon source of gut bacteria; moreover, host circulating androgen levels may be regulated by androgen-metabolizing gut bacteria.

Similar (but not identical) mechanisms and corresponding androgen-transforming enzymes have been identified in the androgen-metabolizing gut bacteria. It is known that dihydrotestosterone significantly inhibits the hair re-growth in vertebrates. By using male mouse as the model organism, we confirmed the improvement of these androgen-metabolizing gut bacteria, especially Bacillus coagulans, in host hair re-growth through reducing the circulating dihydrotestosterone level.

In one embodiment, the androgen-metabolizing gut bacteria can be formulated in a probiotic food or a medicament.

In some embodiments, the medicament is a pharmaceutical composition. The pharmaceutical composition may contain, in addition to the androgen-metabolizing gut bacteria in an effective amount, a pharmaceutical adjuvant such as an antiseptic, a stabilizer, a wetting agent, an emulsification promoter, a salt and/or buffer for control of osmotic pressure, etc. and other therapeutically useful substances and may be prepared into various formulations for oral or parenteral administration according to common methods.

The formulation for oral administration may be, for example, a tablet, a pill, a hard or soft capsule, a liquid, a suspension, an emulsion, a syrup, a powder, a dust, a fine granule, a granule, a pellet, etc. and these formulations may contain, in addition to the active ingredient, a surfactant, a diluent (e.g., lactose, dextrose, sucrose, mannitol, sorbitol, cellulose and glycine), a lubricant (e.g., silica, talc, stearic acid and a magnesium or calcium salt thereof and polyethylene glycol). The tablet may further contain a binder such as magnesium aluminum silicate, starch paste, gelatin, tragacanth, methyl cellulose, sodium carboxymethyl cellulose and polyvinylpyrrolidine and may contain a pharmaceutical additive such as a disintegrant, e.g., starch, agar or alginic acid or a sodium salt thereof, an absorbent, a colorant, a flavor, a sweetener, etc. as occasion demands. The tablet may be prepared by a common mixing, granulation or coating method.

The formulation for parenteral administration may be a formulation for transdermal administration, e.g., an injection, a drip, an ointment, a lotion, a gel, a cream, a spray, a suspension, an emulsion, a suppository, a patch, etc., although not being limited thereto.

Determination of the administration dosage of the active ingredient (such as androgen-metabolizing gut bacteria) is within the level of those of ordinary skill. A daily administration dosage may vary depending on various factors such as the stage of the disease to be treated, age, health condition, presence of complication, etc., However, the administration dosage does not limit the scope of the present disclosure by any means.

The pharmaceutical composition may be a formulation for external application to the skin. The formulation for external application to the skin includes any formulation that can be applied externally on the skin and various types of medical formulations may be included therein. In an exemplary embodiment, the composition may be a cosmetic composition.

When the formulation of the present disclosure is a solution or an emulsion, a solvent, a solubilizer or an emulsifier may be used as a carrier ingredient. For example, water, ethanol, isopropanol, ethyl carbonate, ethyl acetate, benzyl alcohol, benzyl benzoate, propylene glycol, 1,3-butyl glycol oil, a glycerol aliphatic ester, polyethylene glycol or a fatty acid ester of sorbitan may be used.

In an exemplary embodiment, the composition may be a food composition.

The food composition may be a liquid or solid formulation. For example, it may be in the form of various foods, beverages, gums, teas, vitamin mixtures, supplementary health foods, etc. and may be formulated as a powder, a granule, a tablet, a capsule or a drink. Each formulation of the food composition may be prepared by those skilled in the art without difficulty by mixing the active ingredient with ingredients commonly used in the art depending on purposes. A synergic effect may be achieved when the active ingredient is used together with other ingredients.

Liquid ingredients that may be contained in the food composition in addition to the active ingredient are not particularly limited. Various flavors, natural carbohydrates, etc. may be further contained as in common drinks. The natural carbohydrate may be a monosaccharide, a disaccharide such as glucose, fructose, etc., a polysaccharide such as maltose, sucrose, etc., a common sugar such as dextrin, cyclodextrin, etc. or a sugar alcohol such as xylitol, sorbitol, erythritol, etc. As the flavor, a natural flavor (thaumatin, stevia extract (e.g., rebaudioside A, glycyrrhizin, etc.) or a synthetic flavor (e.g., saccharin, aspartame, etc.) may be used. The natural carbohydrate may be contained in an amount of generally about 1-20 g, specifically about 5-12 g, per 100 mL of the composition according to the present disclosure.

In an aspect, the food composition may contain various nutrients, vitamins, minerals (electrolytes), flavors such as synthetic flavors and natural flavors, colorants, extenders (cheese, chocolate, etc.), pectic acid and salts thereof, alginic acid and salts thereof, organic acids, protective colloidal thickeners, pH control agents, stabilizers, antiseptics, glycerin, alcohols, carbonating agents used in carbonated drinks, etc. In another aspect, pulps for preparing natural fruit juice and vegetable drinks may be contained. These ingredients may be used independently or in combination. These additives may be used in an amount of about 0.001-20 parts by weight per 100 parts by weight of the composition according to the present disclosure, although not being limited thereto.

The probiotic may be administered as a daily dose and in the form of a composition. The daily dose of androgen-metabolizing gut bacteria administered is from 1×10⁶ to 1×10¹² cfu, preferably 1×10⁸ to 1×10¹¹ cfu (cfu=colony forming unit).

The above doses include the possibilities that the bacteria are live, inactivated or dead, or even present as fragments such as DNA or cell wall or cytoplasmic materials, or as bacteria fermentation products or as bacteria metabolites. In other words, the quantity of bacteria which the formula contains is expressed in terms of the colony forming ability of that quantity of bacteria as if all the bacteria were live, irrespective of whether they are, in fact, live, inactivated or dead, fragmented, or in the form of fermentation products or metabolites, or a mixture of any or all of these states.

Other examples of synergistic compounds that may be included in the compositions are prebiotic compounds. A prebiotic is a non-digestible food ingredient that beneficially affects the host by selectively stimulating the growth and/or activity of one or a limited number of bacteria in the colon, and thus improves host health. Such ingredients are non-digestible in the sense that they are not broken down and absorbed in the stomach or small intestine and thus pass intact to the colon, where they are selectively fermented by the beneficial bacteria. Examples of prebiotics include certain oligosaccharides, such as fructo-oligosaccharides (FOS), cow milk oligosaccharides (CMOS) and galacto-oligosaccharides (GOS). A combination of prebiotics may be used such as 90% GOS with 10% short chain fructooligosaccharides such as the product sold under the trade mark Raftilosc® or 10% inulin such as the product sold under the trade mark Raftiline®. Other examples of prebiotics that can be used in the context of the present invention include the group of oligosaccharides obtained from milk or other sources, optionally containing sialic acid, fructose, fucose, galactose or mannose. Preferred prebiotics are sialo-oligosaccharides (SOS), fructo-oligosaccharides (FOS), galacto-oligosaccharides (GOS), isomalto-oligosaccharides (IMO), xylo-oligosaccharides (XOS), arabino-xylo oligosaccharides (AXOS), mannan oligosaccharides (MOS), oligosaccharides of soy, glycosylsucrose (GS), lactosucrose (LS), sialyl-lactose (SL), fucosyl-lactose (FL), lacto-N-neotetraose (LNNT), lacto-neotetraose (LNT), lactulose (LA), palatinose-oligosaccharides (PAO), malto-oligosaccharides, gums/or hydrolysates thereof, pectins, starches, and/or hydrolysates thereof. An infant formula according to the invention preferably further contains at least one prebiotic in an amount of 0.3 to 10% of the total weight of the dry composition.

The following experiments and data are provided for illustrating the present disclosure.

### Examples

### Analytical Chemical Methods

The dihydrotestosterone (DHT) degradation activities of bacterial strains were determined through the following chemical techniques:

### (a) Thin-Layer Chromatography (TLC)

The steroid standards, including DHT, and microbial products were separated on a silica gel-coated aluminum TLC plate (Silica gel 60 F₂₅₄: thickness, 0.2 mm; 20×20 cm; Merck®) using dichloromethane: ethyl acetate: ethanol (14:4:0.05, v/v/v) as the developing phase. The steroids were visualized under UV light at 254 nm or by spraying the TLC plates with 30% (v/v) H₂SO₄, followed by an incubation for 1 min at 100° C. (in an oven).

### (b) High-Performance Liquid Chromatography (HPLC)

A reversed-phase HITACHI® HPLC system equipped with an analytical RP-C18 column [Luna 18 (2), 5 μm, 150×4.6 mm; PHENOMENEX®] was used for separating steroid metabolites in this study. The separation was achieved isocratically using a mobile phase of 45% methanol (v/v) at 35° C. at a flow rate of 0.5 mL/min. The steroid metabolites were detected using a photodiode array detector (200-450 nm). In some studies, HPLC was also used for quantifying steroids extracted from the strain cultures. The quantity of major androgens (AD, ADD, DHT, and testosterone) was determined using a standard curve generated from individual steroid standards. The R² values for the standard curves were >0.98. The presented data are the average values of three experimental measurements.

### (c) Ultra-Performance Liquid Chromatography High-Resolution Mass Spectrometry (UPLC HRMS)

The androgen metabolites in mouse fecal (0.1 g) or serum (0.2 mL) samples were extracted using ethyl acetate for three times. Before the extraction, 0.1 ng of [2,3,4C-¹³C]testosterone (internal standard) was added to the samples. After the solvent was evaporated, the residue was re-dissolved in 10 μL of methanol. The androgen metabolites were identified by comparing their corresponding retention time and m/z values to the authentic standards. The androgen metabolites were quantified using the standard curves established using the authentic standards (linear range: 0.05-20 ng/ml; 2-fold serial dilution).

Androgen metabolites were analyzed using UPLC-HRMS on a UPLC system coupled to either an Electric Spray Ionization-Mass Spectrometry (ESI-MS) system or an Atmosphere Pressure Chemical Ionization-Mass Spectrometry (APCI-MS) system. Androgen metabolites were firstly separated using a reversed-phase Cis column (ACQUITY UPLC® BEH C18; 1.7 μm; 100×2.1 mm; Waters) at a flow rate of 0.4 mL/min at 35° C. (oven temperature). The mobile phase comprised a mixture of two solutions: solution A [0.1% formic acid (v/v) in 2% acetonitrile (v/v)] and solution B [0.1% formic acid (v/v) in methanol]. Separation was achieved using a gradient of solvent B from 10% to 99% in 8 min. ESI-HRMS analysis was performed using a THERMO FISHER SCIENTIFIC™ ORBITRAP ELITE™ Hybrid Ion Trap-Orbitrap Mass Spectrometer (WALTHAM®, MA, USA). Mass spectrometric data in positive ionization mode were collected. The source voltage was set at 3.2 kV; the capillary and source heater temperatures were 360° C. and 350° C., respectively; the sheath, auxiliary, and sweep gas flow rates were 30, 15 and 2 arb units, respectively. APCI-HRMS analysis was performed using a THERMO FISHER SCIENTIFIC™ ORBITRAP ELITE™ Hybrid Ion Trap-Orbitrap Mass Spectrometer (Waltham, MA, USA) equipped with a standard APCI source. Mass spectrometric data in positive ionization mode (parent scan range: 50-600 m/z) were collected. The capillary and APCI vaporizer temperatures were 120° C. and 400° C., respectively; the sheath, auxiliary, and sweep gas flow rates were 40, 5 and 2 arbitrary units, respectively. The source voltage was 6 kV and the current was 15 μA. The elemental composition of individual adduct ions was predicted using XCALIBUR™ Software (THERMO FISHER SCIENTIFIC™).

### General Molecular Biological Methods

The bacterial genomic DNA was extracted using the PRESTO™ Mini gDNA Bacteria Kit (GENEAID®, New Taipei City, Taiwan). PCR mixtures (50 μL) contained nuclease-free H₂O, 2×PCR master mix (INVITROGEN™ Platinum™ Hot Start PCR 2X Master Mix, Thermo Fisher Scientific, Waltham, MA, USA), forward and reverse primers (200 nM each), and template DNA (10-30 ng). The PCR products were verified using standard TAE-agarose gel (1.5%) electrophoresis with the SYBR® Green I nucleic acid gel stain (INVITROGEN THERMO FISHER SCIENTIFIC™, Waltham, MA, USA), and the PCR products were purified using the GenepHlow Gel/PCR Kit (GENEAID®, New Taipei City, Taiwan). The TA cloning was performed with T&A™ Cloning Vector Kit (YEASTERN BIOTECH®, New Taipei City, Taiwan).

### Molecular Identification of Bacterial Strains Through PCR

Bacterial purity was checked microscopically and by growth tests in Brain Heart Infusion (BHI) broth medium or BHI agar. The 16S rRNA genes of the four isolated colonies capable of anaerobic DHT degradation in the denitrifying medium were PCR-amplified using the bacterial 16S rRNA universal primers 27F (5′-AGAGTTTGATCCTGGCTCAG-3′, SEQ ID NO: 1) and 1492R (5′-GGTTACCTTGTTACGACTT-3′, SEQ ID NO: 2) (Lane, D. J. (1991). 16S/23S rRNA sequencing. In: Stackebrandt, E., and Goodfellow, M. (eds.). Nucleic acid techniques in bacterial systematics. Chichester, United Kingdom: John Wiley and Sons), and the resulting PCR products were sequenced to verify the identity of the isolates.

### Evaluating the Effects of Potential Probiotics on Hair Regrowth in Mice Model

The animal experiments were approved by the Institutional Animal Care and Use Committee (IACUC No. 20220423). Seven-week-old male C57BL/6J mice were shaved and randomly divided into three groups: vehicle, DHT, and DHT+probiotic. The vehicle group was administered 100 μL of sesame oil and 100 μL of phosphate-buffered saline (PBS), the DHT group received 100 μL of DHT (5 mg) dissolved in sesame oil and 100 μL of PBS, and the DHT+probiotic group was treated with 100 μL of DHT (5 mg) dissolved in sesame oil and 100 μL of probiotics (approximately 5×10⁸ CFU) suspended in PBS. The dosage of DHT administration in mice was followed Fu et. al. (Fu D et. al. (2021). Dihydrotestosterone-induced hair regrowth inhibition by activating androgen receptor in C57BL6 mice simulates androgenetic alopecia. Biomed Pharmacother. 137:111247). Treatments were administered twice per week for 4 weeks by oral gavage. Photographs of the mice were taken from a dorsal view to document hair regrowth, and the hair regrowth area was quantified using ImageJ software.

### Example 1 Novel Androgen-Metabolizing Gut Bacteria

Bacteria BRCAS-C1 to C4 are the strains with higher DHT-metabolizing activity and used for the following testosterone activity test through yeast androgenic assay. To the best of our knowledge, the novel function (anaerobic DHT metabolism and improvement of hair-regrowth) of lactic acid bacteria has not been reported. Moreover, nucleotide sequences of the 16S rRNA genes of the strains BRCAS-CI (SEQ ID NO: 3), BRCAS-C2 (SEQ ID NO: 4), BRCAS-C3 (SEQ ID NO: 5), and BRCAS-C4 (SEQ ID NO: 6) are not completely identical to any available bacterial 16S rRNA genes in the NCBI. Together, the genetic, physiological, and metabolic characterizations of these four bacterial strains indicate their novelty in both metabolic function and genetics. The impact of these bacterial strains in host physiology (e.g., hair regrowth) is also novel.

### Example 2 the Novel Androgen-Metabolizing Gut Bacteria can Reduce the Androgenic Activity of DHT

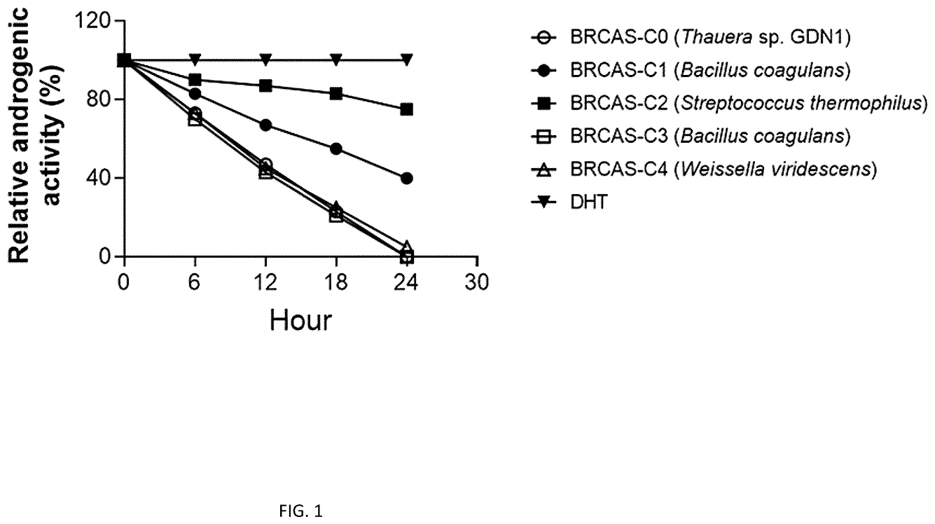

Individual bacterial strains were anaerobically incubated in BHI medium containing 1 mM DHT and bacterial cultures were sampled every 6 days. The steroids in bacterial cultures were extracted with ethyl acetate for three times, and androgenic activity of the ethyl acetate extracts were determined using a yeast androgenic assay. As shown in FIG. 1, BRCAS-C1 to BRCAS-C4 reduce the androgenic activity of DHT, especially BRCAS-C3 and BRCAS-C4.

### Example 3 Administration of the Androgen-Metabolizing Gut Bacteria Alleviates the DHT-Inhibited Hair Loss in Male Mice

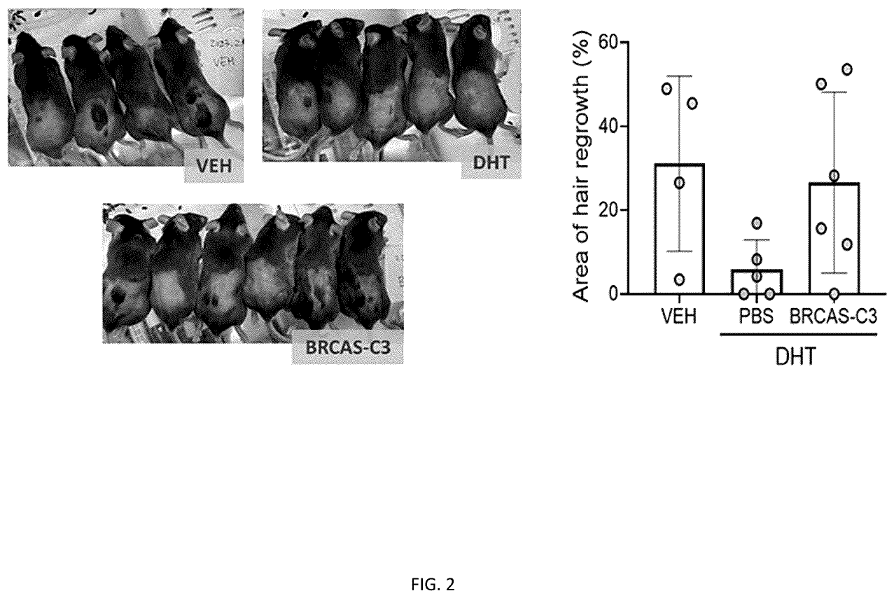

FIG. 2: Hair-shaved mice were administered with basal medium (VEH) alone, basal medium containing DHT, or basal medium containing DHT and strain BRCAS-C3. The tested mice were photographed on Day 28. The areas of re-grown hair were quantified using ImageJ™. As shown in FIG. 2, administration of BRCAS-C3 alleviates the DHT-inhibited hair loss in male mice.

1. A probiotic composition comprising androgen-metabolizing gut bacteria, wherein the androgen-metabolizing gut bacteria comprise Bacillus coagulans and Streptococcus thermophilus, and/or Weissella viridescens and any combination of the above-mentioned bacteria.

2. The probiotic composition of claim 1, wherein the Bacillus coagulans comprises Bacillus coagulans BRCAS-C1 or Bacillus coagulans BRCAS-C3.

3. The probiotic composition of claim 1, wherein the Streptococcus thermophilus comprises Streptococcus thermophilus BRCAS-C2.

4. The probiotic composition of claim 1, wherein the Weissella viridescens comprises Weissella viridescens BRCAS-C4.

5. The probiotic composition of claim 1, wherein the combination of the bacteria comprises any of the following: Bacillus coagulans and Streptococcus thermophilus; Bacillus coagulans and Weissella viridescens; Streptococcus thermophilus and Weissella viridescens; and Bacillus coagulans, Streptococcus thermophilus and Weissella viridescens.

6. The probiotic composition of claim 1, wherein the androgen-metabolizing gut bacteria further comprises Thauera sp.

7. The probiotic composition of claim 6, wherein the combination of the bacteria comprises any of the following: Bacillus coagulans and Thauera sp.; Streptococcus thermophilus and Thauera sp.; Weissella viridescens and Thauera sp.; Bacillus coagulans, Streptococcus thermophiles and Thauera sp.; Bacillus coagulans, Weissella viridescens and Thauera sp.; Streptococcus thermophiles, Weissella viridescens and Thauera sp.; Bacillus coagulans, Streptococcus thermophiles, Weissella viridescens and Thauera sp.

8. The probiotic composition of claim 1, which is used as probiotic food or a medicament.

9. A method for treating or preventing a disease, disorder or condition caused by hyperactivity of a serum androgen level and/or increasing hair re-growth in a subject in need thereof, comprising administering androgen-metabolizing gut bacteria to the subject.

10. The method of claim 9, wherein the method is for regulating the serum androgen level or reducing the circulating dihydrotestosterone level.

11. The method of claim 9, wherein the disease, disorder or condition is androgenetic alopecia, benign prostatic hypertrophy, prostate cancer, polycystic ovary syndrome (PCOS), acne, hirsutism, hyperandrogenism, male infertility, or seborrhea.

12. The method of claim 9, wherein the androgen-metabolizing gut bacteria is formulated in a probiotic food or a medicament.

13. The method of claim 9, wherein the androgen-metabolizing gut bacteria is selected from the group consisting of: Bacillus sp., Streptococcus sp., Weissella sp. and any combination thereof.

14. The method of claim 13, wherein the androgen-metabolizing gut bacteria Bacillus sp. is Bacillus coagulans.

15. The method of claim 13, wherein the Streptococcus sp. is Streptococcus thermophilus.

16. The method of claim 13, wherein the Weissella sp. is Weissella viridescens.

17. The method of claim 9, wherein the combination of the androgen-metabolizing gut bacteria comprises any of the following: Bacillus coagulans and Streptococcus thermophilus; Bacillus coagulans and Weissella viridescens; Streptococcus thermophilus and Weissella viridescens; and Bacillus coagulans, Streptococcus thermophilus and Weissella viridescens.

18. The method of claim 9, wherein the androgen-metabolizing gut bacteria further comprises Thauera sp.

19. The method of claim 18, wherein the combination of the androgen-metabolizing gut bacteria comprises any of the following: Bacillus coagulans and Thauera sp.; Streptococcus thermophilus and Thauera sp.; Weissella viridescens and Thauera sp.; Bacillus coagulans, Streptococcus thermophiles and Thauera sp.; Bacillus coagulans, Weissella viridescens and Thauera sp.; Streptococcus thermophiles, Weissella viridescens and Thauera sp.; Bacillus coagulans, Streptococcus thermophilus and Weissella viridescens and Thauera sp.

20. The method of claim 9, wherein the androgen-metabolizing gut bacteria comprises Bacillus coagulans BRCAS-C1, Bacillus coagulans BRCAS-C3, Streptococcus thermophilus BRCAS-C2, or Weissella viridescens BRCAS-C4.


SEQUENCE LISTING (6 sequences)

SEQ ID NO: 1 - DNA, 20 bp, synthetic construct
  source 1..20: mol_type = other DNA
agagtttgatcctggctcag

SEQ ID NO: 2 - DNA, 19 bp, synthetic construct
  source 1..19: mol_type = other DNA
ggttaccttgttacgactt

SEQ ID NO: 3 - DNA, 1335 bp, Bacillus coagulans
  source 1..1335: mol_type = genomic DNA
ggttagcggcggacgggtgagtaacacgtgggcaacctgcctgtaagatcgggataacgccgggaaaccggggctaataccggatagttttttcctccgcatggaggaaaaaggaaagacggcttykgctgtcacttacagatgggcccgcggcgcattagcttgttggtggggtaacggctcaccaaggcaacgatgcgtagccgacctgagagggtgatcggccacattgggactgagacacggcccaaactcctacgggaggcagcagtagggaatcttccgcaatggacgaaagtctgacggagcaacgccgcgtgagtgaagaaggccttcgggtcgtaaaactctgttgccggggaagaacaagtgccgttcgaacagggcggcgccttgacggtacccggccagaaagccacggctaactacgtgccagcagccgcggtaatacgtaggtggcaagcgntgtccggaattattgggcgtaaagcgcgcgcaggcggcttcttaagtctgatgtgaaatcttgcggctcaaccgcaagcggtcattggaaactgggaggcttgagtgcagaagaggagagtggaattccacgtgtagcggtgaaatgcgtagagatgtggaggaacaccagtggcgaaggcggctctctggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaanggaattgacgggggcccgcacaagcggtggagcatgtggtttaattcgaagcaacgcgaagaaccttaccaggtcttgacatcctctgacctccctggagacagggccttccccttcgggggacagagtgacaggtggtgcatggttgtcgtcagctcgtgtcgtgagatgttgggttaagtcccgcaacgagcgcaacccttgaccttagttgccagcattcagttgggcactctaaggtgactgccggtgacaaaccggaggaaggtggggatgacgtcaaatcatcatgccccttatgacctgggctacacacgtgctacaatggatggtacaaagggctgcgagaccgcgaggttaagccaatcccagaaaaccattcccagttcggattgcaggctgcaacccgcctgcatgaagccggaatcgctagtaatcgcggatcagcatgccgcggtgaatacgttcccgggccttgtacacaccgcccgtcacaccacgaga

SEQ ID NO: 4 - DNA, 1544 bp, Streptococcus thermophilus
  source 1..1544: mol_type = genomic DNA
aatgagagtttgatcctggctcaggacgaacgctggcggcgtgcctaatacatgcaagtagaacgctgaagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaaggtgcggctggatcacctcctt

SEQ ID NO: 5 - DNA, 1552 bp, Bacillus coagulans
  source 1..1552: mol_type = genomic DNA
tactgagagtttgatcctggctcaggacgaacgctggcggcgtgcctaatacatgcaagtcgtgcggaccttttaaaagcttgcttttaaaaggttagcggcggacgggtgagtaacacgtgggcaacctgcctgtaagatcgggataacgccgggaaaccggggctaataccggatagttttttcctccgcatggaggaaaaaggaaagacggcttttgctgtcacttacagatgggcccgcggcgcattagctagttggtggggtaacggctcaccaaggcaacgatgcgtagccgacctgagagggtgatcggccacattgggactgagacacggcccaaactcctacgggaggcagcagtagggaatcttccgcaatggacgaaagtctgacggagcaacgccgcgtgagtgaagaaggccttcgggtcgtaaaactctgttgccggggaagaacaagtgccgttcgaacagggcggtgccttgacggtacccggccagaaagccacggctaactacgtgccagcagccgcggtaatacgtaggtggcaagcgttgtccggaattattgggcgtaaagcgcgcgcaggcggtttcttaagtctgatgtgaaatcttgcggctcaaccgcaagcggtcattggaaactgggaggcttgagtgcagaagaggagagtggaattccacgtgtagcggtgaaatgcgtagagatgtggaggaacaccagtggcgaaggcggctctctggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacctccctggagacagggccttccccttcgggggacagagtgacaggtggtgcatggttgtcgtcagctcgtgtcgtgagatgttgggttaagtcccgcaacgagcgcaacccttgaccttagttgccagcattgagttgggcactctaaggtgactgccggtgacaaaccggaggaaggtggggatgacgtcaaatcatcatgccccttatgacctgggctacacacgtgctacaatggatggtacaaagggctgcgagaccgcgaggttaagccaatcccagaaaaccattcccagttcggattgcaggctgcaacccgcctgcatgaagccggaatcgctagtaatcgcggatcagcatgccgcggtgaatacgttcccgggccttgtacacaccgcccgtcacaccacgagagtttgtaacacccgaagtcggtgaggtaacctttacggagccagccgccgaaggtgggacagatgattggggtgaagtcgtaacaaggtagccgtatcggaaggtgcggctggatcacctccttt

SEQ ID NO: 6 - DNA, 1573 bp, Weissella viridescens
  source 1..1573: mol_type = genomic DNA
atgagagtttgatcctggctcaggatgaacgctggcggcgtgcctaatacatgcaagtcgaacgctttgtggtccaactgatttgaagagcttgctcagatatgacgatggacattgcaaagagtggcgaacgggtgagtaacacgtgggaaacctacctcttagcaggggataacacttggaaacaagtgctaataccgtataacactaataaccgcatggttattagttaaaagatggtcttgctatcactaagagatggtcccgcggtgtattagctagttggtaaggtaatggcttaccaaggcaatgatacatagccgagttgagagactgatcggccacaatgggactgagacacggcccatactcctacgggaggcagcagtagggaatcttccacaatggacgaaagtctgatggagcaacgccgcgtgtgtgatgaagggtttcggctcgtaaaacactgttgtaagagaagaatgacattgagagtaactgttcagtgtgtgacggtatcttaccagaaaggaacggctaaatacgtgccagcagccgcggtaatacgtatgttccaagcgttatccggatttattgggcgtaaagcgagcgcagacggttatttaagtccgaagtgaaagcccacagcttaactgtggaagtgctttggaaactggataacttgagtgcagtagaggagagtggaactccatgtgtagcggtgaaatgcgtagatatatggaagaacaccagtggcgaaggcggctctctggactgtaactgacgttgaggctcgaaagtgtgggtagcaaacaggattagataccctggtagtccacaccgtaaacgatgagtgctagatgtttgagggtttccgcccttaagtgtcgcagctaacgcattaagcactccgcctggggagtacgaccgcaaggttgaaactcaaaggaattgacggggacccgcacaagcggtggagcatgtggtttaattcgaagcaacgcgaagaaccttaccaggtcttgacatcctttgaccacttcagagatgaagctttcccttcggggacaaagtgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggatgacgtcaaatcatcatgccccttatgacctgggctacacacgtgctacaatggcaagtacaacgagcagctaacccgcgagggtacgcgaatctcttaaaacttgtctcagttcggattgtaggctgcaactcgcctacatgaagtcggaatcgctagtaatcgcggatcagcacgccgcggtgaatacgttcccgggtcttgtacacaccgcccgtcacaccatgagagtttgtaacacccaaagccggtgaggtaaccttttaggagccaaccgtctaaggtgggacagatgattagggtgaagtcgtaacaaggtagccgtaggagaacctgcggctggatcacctcctt